(19) United States
(12) Patent Application Publication
(10) Pub. No.: US 2006/0059608 A1
(43) Pub. Date: Mar. 23, 2006

(54) Ventilated eyewear

(75) Inventor: Mario Difilippo, Asolo (IT)

Correspondence Address: WOLF GREENFIELD & SACKS, PC;FEDERAL RESERVE PLAZA, 600 ATLANTIC AVENUE, BOSTON, MA, 02210-2211, US

(73) Assignee: The Burton Corporation, Burlington, VT (US)

(21) Appl. No.: 11/213,312

(22) Filed: Aug. 26, 2005

Related U.S. Application Data
(60) Provisional application No. 60/604,767, filed on Aug. 26, 2004.

(51) Int. Cl. A61F 9/02

(52) U.S. Cl. 2/436

(57) ABSTRACT

Ventilated eyewear comprising a lens and a frame having one or more openings incorporating breathable fabric. Air and/or moisture can pass through the fabric in the openings to ventilate the space between the eyewear and the wearer's face.

### RELATED APPLICATIONS

This patent application claims priority to U.S. Provisional Application Ser. No. 60/604,767, filed Aug. 26, 2004, which is hereby incorporated by reference in its entirety.

### BACKGROUND OF INVENTION

1. Field of Invention

The invention is directed to eyewear and, more particularly, to a goggle such as a ventilated snowsports goggle.

2. Discussion of Related Art

Goggles are used in many applications to protect a wearer's eyes from the environment. In industry, goggles may be employed to prevent injury to a worker's eyes. In various sports, goggles may be worn for safety and/or to help maintain visibility that might be compromised by weather conditions, such as rain, snow, wind, sunlight and/or glare. Representative is a snowsports goggle, that is worn when skiing or snowboarding. Such snowsports goggles typically include a frame, a lens or a pair of lenses, and a strap for securing the goggle to a wearer's helmet or head. Ventilation is important to prevent the lens from fogging up which may distort or reduce visibility. Typically, small perforations or slots are provided in a top, bottom or side piece of the frame, allowing air to circulate across the inner face of the lens, reducing the likelihood of fogging and misting.

### SUMMARY OF INVENTION

In one embodiment of the invention, ventilated eyewear includes a lens and a frame supporting the lens, the frame and the lens defining an interior space. One or more ventilation openings, such as a slot, are provided in the frame. The ventilation opening provides a pathway between the interior space and an environment that is external to the eyewear when worn by a user. A breathable fabric, such as a mesh, is incorporated at the one or more ventilation openings. By incorporated, it is meant that the fabric may extend over, under and/or within the ventilation opening. The ventilation opening and the breathable fabric allow ventilation of the interior space to the external environment. The breathable fabric may be water-resistant. The breathable fabric may include one or more layers, and each layer in a multi-layer arrangement may be formed of the same or different materials as compared to another layer. In certain embodiments, an individual layer may be formed of more than one material. Perforations may be formed into and/or through the breathable fabric. The breathable fabric may include a coating or other treatment to improve durability, waterproofing, and/or performance properties.

In another embodiment of the invention, the ventilated eyewear is a goggle, preferably a snowsports goggle, including a frame, at least one lens, one or more ventilation openings in the frame, and a breathable fabric incorporated at such ventilation openings. Ventilation openings may be located at a top portion of the frame, a first side of the frame, and/or a second side of the frame opposed to the first side. The one or more ventilation openings and the breathable fabric allow ventilation of the interior space to the external environment. A face gasket may be provided with the frame to contact a user's face when the goggles are worn. A strap, preferably adjustable, may be provided to secure the goggle to a user, such as about the user's head whether uncovered or if wearing a hat, helmet or other head covering.

### BRIEF DESCRIPTION OF DRAWINGS

The accompanying drawings, are not intended to be drawn to scale. In the drawings, each identical or nearly identical component that is illustrated in various figures is represented by a like numeral. For purposes of clarity, not every component may be labeled in every drawing. In the drawings:

FIGS. 1(a)-(c) are a top view, a side view, and a partially angled perspective view of the eyewear according to the present invention.

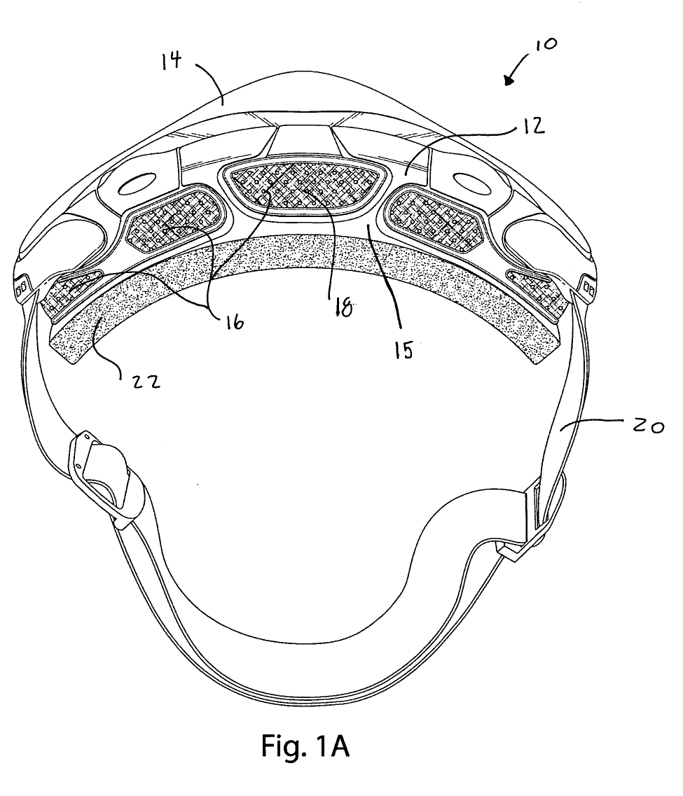

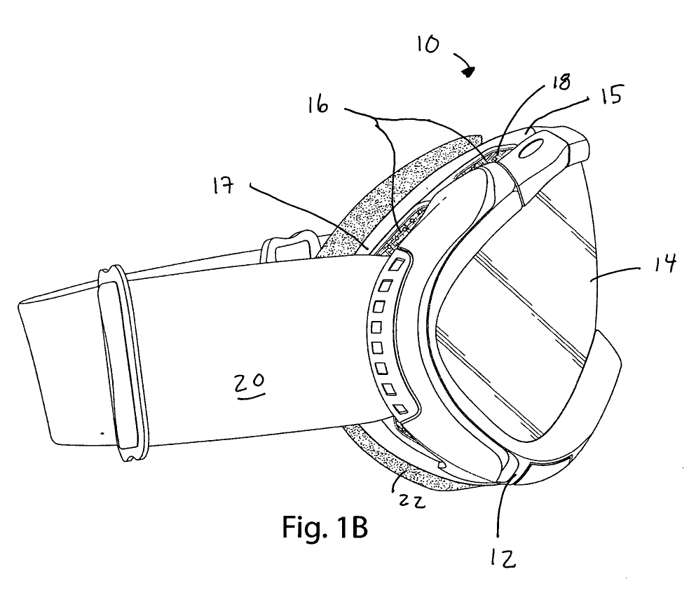

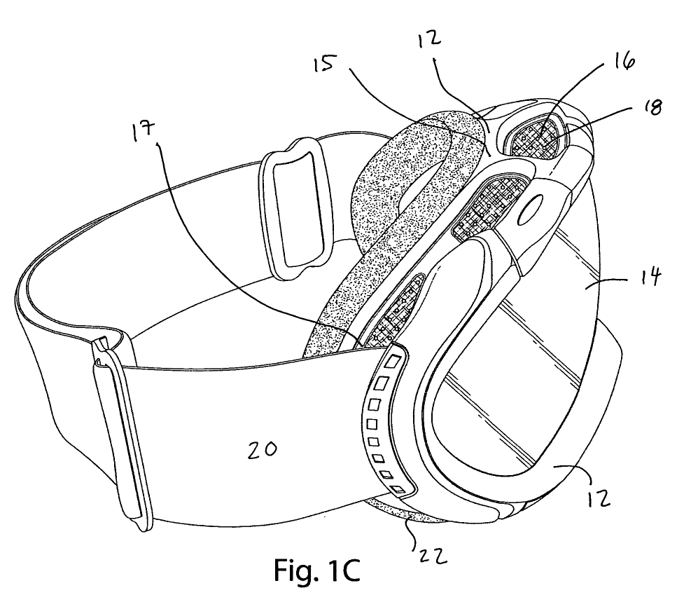

### DETAILED DESCRIPTION

This invention is not limited in its application to the details of construction and the arrangement of components set forth in the following description or illustrated in the drawings. The invention is capable of other embodiments and of being practiced or of being carried out in various ways.

The ventilated eyewear of the present invention is described in connection with a snowsports goggle. However, the invention is not so limited, and it is contemplated that the ventilated eyewear also may by employed in other types of goggles, as an eyeglass or a sunglass, to name but a few other applications.

As shown in the drawings, a ventilated eyewear 10 includes a frame 12 for holding a single lens 14 for both the right and left eyes, or a pair of lenses with each lens specific to one eye. The frame includes one or more openings or vents 16, such as the slots provided in the top 15 and side 17 aspects of the snowsports goggle frame shown in the figures. The size, shape and location of the one or more ventilation openings is not critical to the invention. Extending across the slots in the goggle frame is a breathable fabric 18 that allows air to pass into and/or out of the goggle space. In one embodiment, the breathable fabric is a mesh fabric. To increase air flow, the fabric may further include perforations, such as may be formed by punching a needle through the fabric. The breathable fabric may be woven, knitted, or some other non-sponge or non-foam type fabric. The breathable fabric may include one or more layers of material. The breathability properties may vary as between layers. Further, the number of layers, and composition of the layers, may vary at different vent locations about the frame.

The breathable fabric may extend as a single continuous strip along two or more vent slots, may consist of a single strip that is incorporated at a single vent opening, or may consist of two or more strips at a single vent opening. In one embodiment, the frame is formed in sections that can be joined together. In some of such embodiments, the frame sections include inner and outer sections that can be joined to sandwich the breathable fabric there between, so as to incorporate the fabric at the vent openings. Either or both of the inner and outer sections may further include one or more supporting struts that extend across, above, below, or through a vent opening. A strut may be made of the same material or a different material as the inner and/or outer sections of the frame. In one embodiment, the inner and outer sections are separate structures that are united together such as by adhesive, welding, molding, or mechanical fixation. In other embodiments, a breathable fabric strip may be insert molded to the frame, resulting in the formation of an inner and outer section thereabout.

The breathable fabric may include selected stiffer or reinforcing portions, such as a solid plastic rim extending about the margins of a fabric strip, and/or a solid section extending from edge to edge across the strip, both of which are illustrated in the drawings. The reinforcing material may be applied onto a surface of the fabric, embedded within the fabric, and or extend partially or completely through the fabric. The stiffer section may be formed by other arrangements, as should be apparent to one of skill in the art including, without limitation, by heat setting the fabric; that is, without adding any material to the fabric. The stiffer section need not be solid; that is the stiffer section may, too, be porous and also may be formed of a breathable material.

The goggle may include a strap 20, as shown, for maintaining the goggle in position about the user's head and/or helmet. The strap may be formed of a stretchable or unstretchable fabric and may be adjustable. Alternatively, the strap may be formed of a cord-like arrangement which, too, may be adjustable. The lens may be clear or tinted depending upon the desired application and performance properties. Decentered and/or toroidal lenses may be employed, as may other lens arrangements that are designed for purposes of clarity, UV radiation resistance, and defogging. The type of lens employed is not a critical feature of the invention. The goggle may further include a face foam 22 or other gasket type arrangement around all or at least a portion of the periphery of the frame, to help maintain contact between the goggle and the wearer's face.

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

What is claimed is:

1. Ventilated eyewear comprising:
a lens;
a frame including said lens, said frame and said lens defining an interior space;
a ventilation opening in said frame, said ventilation opening providing a pathway between the interior space and an environment that is external to said eyewear when worn by a user; and
a breathable fabric incorporated at said ventilation opening, said ventilation opening and said breathable fabric allowing ventilation of the interior space to external environment.

2. The ventilated eyewear of claim 1, wherein said ventilation opening is positioned at a top portion of said frame.

3. The ventilated eyewear of claim 1, wherein said ventilation opening is positioned at a lateral side of said eyewear.

4. The ventilated eyewear of claim 1, wherein said ventilation opening includes a plurality of openings.

5. The ventilated eyewear of claim 4, wherein at least one of said plurality of openings is positioned on each of a first side of said frame, a second side of said frame opposed to said first side, and a top portion of said frame.

6. The ventilated eyewear of claim 1, wherein said breathable fabric comprises first and second layers of fabric, said first and said second layers including different fabric materials.

7. The ventilated eyewear of claim 1, further comprising:
a strut at said ventilation opening.

8. The ventilated eyewear of claim 7, wherein said strut extends at least partially across said ventilation opening.

9. The ventilated eyewear of claim 8, wherein said breathable fabric includes first and second layers of fabric and said strut is positioned between said first and said second layers.

10. The ventilated eyewear of claim 9, wherein said first and said second layers of fabric include different fabric materials.

11. The ventilated eyewear of claim 1, wherein said breathable fabric is perforated.

12. The ventilated eyewear of claim 1, wherein said breathable fabric includes a mesh fabric.

13. The ventilated eyewear of claim 11, wherein said breathable fabric includes a coating to improve at least one of water proofing or durability of said breathable fabric.

14. The ventilated eyewear of claim 1, wherein said breathable fabric is insert molded to said frame.

15. The ventilated eyewear of claim 1, wherein said eyewear is a snowsports goggle.

16. The ventilated eyewear of claim 1, further comprising:
a face gasket attached to said frame, said face gasket adapted to contact a user's face when said eyewear is worn.

17. The ventilated eyewear of claim 16, wherein said face gasket includes a foam material.

18. The ventilated eyewear of claim 1, further comprising:
an adjustable strap.

19. The ventilated eyewear of claim 18, wherein said adjustable strap is formed of multiple cords.

20. A ventilated snowsports goggle comprising:
a lens;
a frame including said lens, said frame and said lens defining an interior space;
a ventilation opening in said frame, said ventilation opening providing a pathway between the interior space and an environment that is external to said goggle when worn by a user;
a breathable fabric incorporated at said ventilation opening, said ventilation opening and said breathable fabric allowing ventilation of the interior space to the external environment;
a face gasket attached to said frame, said face gasket adapted to contact a user's face when said goggle is worn; and
an adjustable strap adapted to position said goggle to a user.

21. The ventilated snowsports goggle of claim 20, wherein said ventilation opening is positioned at a top portion of said frame.

22. The ventilated snowsports goggle of claim 20, wherein said ventilation opening is positioned at a lateral side of said goggle.

23. The ventilated snowsports goggle of claim 20, wherein said ventilation opening includes a plurality of openings.

24. The ventilated snowsports goggle of claim 23, wherein at least one of said plurality of openings is positioned on each of a first side of said frame, a second side of said frame opposed to said first side, and a top portion of said frame.

25. The ventilated snowsports goggle of claim 20, wherein said breathable fabric comprises first and second layers of fabric, said first and said second layers including different fabric materials.

26. The ventilated snowsports goggle of claim 20, further comprising:
a strut at said ventilation opening.

27. The ventilated snowsports goggle of claim 26, wherein said strut extends at least partially across said ventilation opening.

28. The ventilated snowsports goggle of claim 27, wherein said breathable fabric includes first and second layers of fabric and said strut is positioned between said first and said second layers.

29. The ventilated snowsports goggle of claim 28, wherein said first and said second layers of fabric include different fabric materials.

30. The ventilated snowsports goggle of claim 20, wherein said breathable fabric is perforated.

31. The ventilated snowsports goggle of claim 20, wherein said breathable fabric includes a mesh fabric.

32. The ventilated snowsports goggle of claim 31, wherein said breathable fabric includes a coating to improve at least one of water proofing or durability of said breathable fabric.

33. The ventilated snowsports goggle of claim 20, wherein said breathable fabric is insert molded to said frame.

34. The ventilated snowsports goggle of claim 20, wherein said face gasket includes foam material.

35. The ventilated snowsports goggle of claim 20, wherein said adjustable strap is formed of multiple cords.
